(19) United States
(12) Patent Application Publication
(10) Pub. No.: US 2024/0285332 A1
(43) Pub. Date: Aug. 29, 2024

(54) TEMPERATURE REGULATING DEVICES AND RELATED SYSTEMS AND METHODS

(71) Applicant: NeuWave Medical, Inc., Madison, WI (US)

(72) Inventors: Jonathan Duff, Madison, WI (US); Jason Brunkow, Madison, WI (US); Christopher Unseth, Madison, WI (US); Emily Foran, Madison, WI (US); Mark Thom, Madison, WI (US)

(21) Appl. No.: 18/113,991

(22) Filed: Feb. 24, 2023

(51) Int. Cl. A61B 18/14; A61B 18/18

(52) U.S. Cl. CPC A61B 18/14; A61B 18/1815; A61B 2017/00221; A61B 2017/00955; A61B 2018/00017; A61B 2018/00035; A61B 2018/00577; A61B 2018/00797; A61B 2018/00821

(57) ABSTRACT

The present invention relates to devices capable of a) measuring a temperature at or near the device (e.g., a tissue region in contact with the device), and b) regulating the temperature (e.g., increasing, maintaining, or reducing) at or near the device, and related systems and methods.

### FIELD OF INVENTION

The present invention relates to devices capable of a) measuring a temperature at or near the device (e.g., a tissue region in contact with the device), and b) regulating the temperature (e.g., increasing, maintaining, or reducing) at or near the device, and related systems and methods.

### BACKGROUND

Medical procedures involving delivery of ablation energy can result in undesired tissue burning (e.g., skin, organ, non-targeted tissue region). Current coolant methods (e.g., water, CO₂) do not provide adequate temperature regulation to prevent or mitigate undesired tissue burning.

The present invention addresses this need.

### SUMMARY

The present invention relates to devices capable of a) measuring a temperature at or near the device (e.g., a tissue region in contact with the device), and b) regulating the temperature (e.g., increasing, maintaining, or reducing) at or near the device, and related systems and methods.

In certain embodiments, the present invention provides a device comprising:
an elongate tubular body having an elongate tubular body interior region, elongate tubular body exterior region, an elongate tubular body proximal end, and elongate tubular body distal end;
wherein the elongate tubular body defines an elongate tubular body linear axis of the device extending from the elongate tubular body proximal end to the elongate tubular body distal end;
wherein the elongate tubular body exterior region has thereon one or more temperature sensors, wherein each of the one or more temperature sensors is configured to measure a temperature of the elongate tubular body exterior region in the vicinity of the specific temperature sensor;
wherein the elongate tubular body interior region has therein one or more coolant channels for circulating and recirculating coolant.

In some embodiments, the device further comprises a stylet tip attached at the elongate tubular body distal end.

In some embodiments, the elongate tubular body is flexible. In some embodiments, the elongate tubular body comprises plastic (e.g., PEEK). In some embodiments, the elongate tubular body comprises thermoplastic polymers that have an appropriate glass-transition temperature of approximately 15-25 degrees Celsius. In some embodiments, the thermoplastic polymer comprises copolymers of lactic acid and caprolactone, wherein the thermoplastic polymer comprises a copolymer of L-lactide and caprolactone such as poly(L-lactide-co-caprolactone) with an L-lactide to caprolactone monomer ratio of 70:30 or less.

In some embodiments, the one or more coolant channels are configured to reduce the temperature of specific regions of the elongate tubular body exterior region or the entire elongate tubular body exterior region.

In some embodiments, each of the one or more coolant channels is separately paired with a separate temperature sensor such that circulation of coolant through the specific coolant channel results in a reduction of temperature of the elongate tubular body exterior region at and/or in the vicinity of the temperature sensor for which it is paired.

In some embodiments, the one or more temperature sensors comprises a first temperature sensor, and the one or more coolant channels comprises a first coolant channel. In such embodiments, the first coolant channel is positioned such that circulation of coolant through the first coolant channel results in a reduction of temperature of the elongate tubular body exterior region at and/or in the vicinity of the first temperature sensor.

In some embodiments, the one or more temperature sensors comprises a first temperature sensor and a second temperature sensor, and the one or more coolant channels comprises a first coolant channel and a second coolant channel. In such embodiments, the first coolant channel is positioned such that circulation of coolant through the first coolant channel results in a reduction of temperature of the elongate tubular body exterior region at and/or in the vicinity of the first temperature sensor; and the second coolant channel is positioned such that circulation of coolant through the second coolant channel results in a reduction of temperature of the elongate tubular body exterior region at and/or in the vicinity of the second temperature sensor.

In some embodiments, the one or more temperature sensors comprises a first temperature sensor, a second temperature sensor, and a third temperature sensor, and the one or more coolant channels comprises a first coolant channel, a second coolant channel, and a third coolant channel. In such embodiments, the first coolant channel is positioned such that circulation of coolant through the first coolant channel results in a reduction of temperature of the elongate tubular body exterior region at and/or in the vicinity of the first temperature sensor; the second coolant channel is positioned such that circulation of coolant through the second coolant channel results in a reduction of temperature of the elongate tubular body exterior region at and/or in the vicinity of the second temperature sensor; and the third coolant channel is positioned such that circulation of coolant through the third coolant channel results in a reduction of temperature of the elongate tubular body exterior region at and/or in the vicinity of the third temperature sensor.

In some embodiments, the one or more temperature sensors comprises a first temperature sensor, a second temperature sensor, a third temperature sensor, and a fourth temperature sensor, and the one or more coolant channels comprises a first coolant channel, a second coolant channel, a third coolant channel, and a fourth coolant channel. In such embodiments, the first coolant channel is positioned such that circulation of coolant through the first coolant channel results in a reduction of temperature of the elongate tubular body exterior region at and/or in the vicinity of the first temperature sensor; the second coolant channel is positioned such that circulation of coolant through the second coolant channel results in a reduction of temperature of the elongate tubular body exterior region at and/or in the vicinity of the second temperature sensor; the third coolant channel is positioned such that circulation of coolant through the third coolant channel results in a reduction of temperature of the elongate tubular body exterior region at and/or in the vicinity of the third temperature sensor; and the fourth coolant channel is positioned such that circulation of coolant through the fourth coolant channel results in a reduction of temperature of the elongate tubular body exterior region at and/or in the vicinity of the fourth temperature sensor.

In some embodiments, the one or more temperature sensors comprises a first temperature sensor, a second temperature sensor, a third temperature sensor, a fourth temperature sensor, and a fifth temperature sensor, and the one or more coolant channels comprises a first coolant channel, a second coolant channel, a third coolant channel, a fourth coolant channel, and a fifth coolant channel. In such embodiments, the first coolant channel is positioned such that circulation of coolant through the first coolant channel results in a reduction of temperature of the elongate tubular body exterior region at and/or in the vicinity of the first temperature sensor; the second coolant channel is positioned such that circulation of coolant through the second coolant channel results in a reduction of temperature of the elongate tubular body exterior region at and/or in the vicinity of the second temperature sensor; the third coolant channel is positioned such that circulation of coolant through the third coolant channel results in a reduction of temperature of the elongate tubular body exterior region at and/or in the vicinity of the third temperature sensor; the fourth coolant channel is positioned such that circulation of coolant through the fourth coolant channel results in a reduction of temperature of the elongate tubular body exterior region at and/or in the vicinity of the fourth temperature sensor; the fifth coolant channel is positioned such that circulation of coolant through the fifth coolant channel results in a reduction of temperature of the elongate tubular body exterior region at and/or in the vicinity of the fifth temperature sensor.

In some embodiments, the one or more coolant channels is one coolant channel capable of reducing the temperature of the elongate tubular body exterior at multiple locations. For example, in some embodiments, such a coolant channel has multiple exit port regions configured to reduce the temperature of the elongate tubular body exterior at multiple locations. In some embodiments, such a coolant channel is configured to reduce the temperature of the elongate tubular body exterior at one or more locations. In some embodiments, such a coolant channel is configured to reduce the temperature of the entire elongate tubular body exterior. In some embodiments, the elongate tubular body interior region includes two or more of such coolant channels (e.g., coolant channel capable of reducing the temperature of the elongate tubular body exterior at multiple locations).

In some embodiments, the one or more coolant channels are configured to circulate a pressurized gas. In some embodiments, the pressurized gas is CO₂. In some embodiments, the one or more coolant channels are configured to circulate a pressurized gas at zero to 1000 psi. In some embodiments, the one or more coolant channels are configured to circulate a liquid. In some embodiments, the liquid is water.

In some embodiments, each of the one or more temperature sensors are linearly positioned along the length of the elongate tubular body exterior region. In some embodiments, each of the one or more temperature sensors is less than one centimeter in length along the length of the elongate tubular body. In some embodiments, each of the one or more temperature sensors wraps around the entirety of the elongate tubular body exterior region. In some embodiments, the one or more temperature sensors comprise a thermocouple.

In some embodiments, the one or more temperature sensors comprise a temperature sensor array (e.g., Fiber Bragg Grating optical fiber sensor).

In some embodiments, the diameter of the device is approximately 3 mm or less. In some embodiments, the diameter of the device is 2 mm or less. In some embodiments, the diameter of the device is approximately 1.4 mm or less. In some embodiments, the size of the device is 15 gauge or smaller.

In some embodiments, the one or more temperature sensors are configured to send measured temperature information to a processor. In some embodiments, the one or more temperature sensors are configured to wirelessly send measured temperature information to a processor.

In some embodiments, the processor comprises software that, when executed, causes the processor to manually or automatically:
process temperature information received from the one or more temperature sensors,
identify temperature sensors measuring a temperature of the elongate tubular body exterior region in the vicinity of the specific temperature sensor to be above a pre-determined level,
identify temperature sensors measuring a temperature of the elongate tubular body exterior region in the vicinity of the specific temperature sensor to be at or below a pre-determined level,
direct the device to reduce the temperature of the elongate tubular body exterior region in the vicinity of a specific temperature sensor identified as measuring a temperature of the elongate tubular body exterior region in the vicinity of the specific temperature sensor to be above a pre-determined level.

In some embodiments, the processor comprises software that, when executed, causes the processor to manually or automatically:
process temperature information received from the one or more temperature sensors,
identify temperature sensors measuring a temperature of the elongate tubular body exterior region in the vicinity of the specific temperature sensor to be above a pre-determined level,
identify temperature sensors measuring a temperature of the elongate tubular body exterior region in the vicinity of the specific temperature sensor to be at or below a pre-determined level,
direct the device to reduce the temperature of the elongate tubular body exterior region.

In some embodiments, the processor comprises software that, when executed, causes the processor to manually or automatically:
direct the device to reduce the temperature of the elongate tubular body exterior region.

In certain embodiments, the present invention provides systems including the devices of the present invention and one or more of
a) a processor comprising software that, when executed, causes the processor to manually or automatically:
process temperature information received from the one or more temperature sensors,
identify temperature sensors measuring a temperature of the elongate tubular body exterior region in the vicinity of the specific temperature sensor to be above a pre-determined level,
identify temperature sensors measuring a temperature of the elongate tubular body exterior region in the vicinity of the specific temperature sensor to be at or below a pre-determined level,
direct the device to reduce the temperature of the elongate tubular body exterior region in the vicinity of a specific temperature sensor identified as measuring a temperature of the elongate tubular body exterior region in the vicinity of the specific temperature sensor to be above a pre-determined level;
b) a processor comprising software that, when executed, causes the processor to manually or automatically:
direct the device to reduce the temperature of the elongate tubular body exterior region;
c) a processor comprising software that, when executed, causes the processor to manually or automatically:
process temperature information received from the one or more temperature sensors,
identify temperature sensors measuring a temperature of the elongate tubular body exterior region in the vicinity of the specific temperature sensor to be above a pre-determined level,
identify temperature sensors measuring a temperature of the elongate tubular body exterior region in the vicinity of the specific temperature sensor to be at or below a pre-determined level,
direct the device to reduce the temperature of the elongate tubular body exterior region;
d) a coolant supply in communication with said device; and
e) a power supply electrically connected to the device.

In certain embodiments, the present invention provides methods including providing a device and processor as described herein, positioning the device in a tissue region such that the one or more temperature sensors are positioned to measure the temperature of the elongate tubular body exterior region in the vicinity of each specific temperature sensor; measuring the temperature of the tissue region with the positioned device;
identifying temperature sensors measuring a temperature of the elongate tubular body exterior region in the vicinity of the specific temperature sensor to be above a pre-determined level,
directing the device to reduce the temperature of the elongate tubular body exterior region.

In some embodiments, directing the device to reduce the temperature of the elongate tubular body exterior region is comprise reducing the temperature of the elongate tubular body exterior region in the vicinity of a specific temperature sensor identified as measuring a temperature of the elongate tubular body exterior region in the vicinity of the specific temperature sensor to be above a pre-determined level. In some embodiments, the tissue region is within a living human being. In some embodiments, the tissue region is at or near an internal organ of a living mammal (e.g., living human being). In some embodiments, the tissue region is skin tissue of a living mammal (e.g., living human being).

Additional embodiments are described herein.

### BRIEF DESCRIPTION OF THE DRAWINGS

FIG. 1 shows temperature regulating device embodiment.

FIG. 2 shows temperature regulating device embodiment.

FIG. 3 shows a coolant channel embodiment for a temperature regulating device.

FIG. 4 shows a temperature regulating device having a cooling zone generated by a coolant channel configured to decrease the temperature at or near a specific location of elongate tubular body exterior region.

FIG. 5 shows a temperature regulating device having three overlapping cooling zones generated by three separate coolant channels configured to decrease the temperature at or near a specific location of elongate tubular body exterior region.

### DETAILED DESCRIPTION

During medical procedures involving tissue ablation with ablation devices (e.g., microwave ablation devices) (e.g., radiofrequency ablation devices) ablation device placement (e.g., positioning) and the targeted ablation region are approximated using imaging and historical ablation data. A significant undesired side effect is the burning of tissue outside of the targeted ablation region. Indeed, when ablating near critical structures it can be difficult to encompass an intended target without damaging the critical structures. The present invention addresses this problem through providing temperature regulating devices capable of a) measuring a temperature at or near the device (e.g., a tissue region in contact with the device), and b) regulating the temperature (e.g., increasing, maintaining, or reducing) at or near the device, and related systems and methods. For example, such temperature regulating devices can be used during an ablation procedure for purposes of monitoring tissue regions outside of the targeted ablation region and ensuring that such tissue regions do not experience tissue burning.

The temperature regulating devices of the present invention are not limited to particular size dimensions. Indeed, in some embodiments, the size dimension of the temperature regulating device is such that it is able to fit within and pass through the lumen of a primary catheter (e.g., an endoscope). In some embodiments, the size dimension of the temperature regulating device is such that it is able to be percutaneously inserted into a living mammal (e.g., living human being), and positioned at internal tissue region within the living mammal. In some embodiments, the diameter of the temperature regulating device is less than 5 mm (e.g., 5 mm or less, 4 mm or less, 3 mm or less, 2 mm or less, 1.4 mm or less, etc.). In some embodiments, the temperature regulating device is of sufficient length to extend from an insertion site (e.g. mouth, incision into body of subject, etc.) to a desired target region within a living body (e.g. 50 cm . . . 75 cm . . . 1 m . . . 1.5 m . . . 2 m . . . 10 m . . . 25 m, etc.). In some embodiments, the flexible sheath is of sufficient length to extend through and beyond the reach of a primary catheter (e.g., endoscope) to reach a treatment site (e.g. peripheral lung tissue, heart tissue, gastrointestinal tissue, etc.) (e.g., any desired location within a living body).

The temperature regulating devices of the present invention are not limited to a particular manner of navigation through a primary catheter and/or through a body region. In some embodiments, the temperature regulating devices comprise a navigation and/or steering mechanism. In some embodiments, the temperature regulating device is without an independent means of navigation, position recognition, or maneuvering. In some embodiments, the temperature regulating device relies upon the primary catheter (e.g., endoscope) or a steerable navigation catheter for placement.

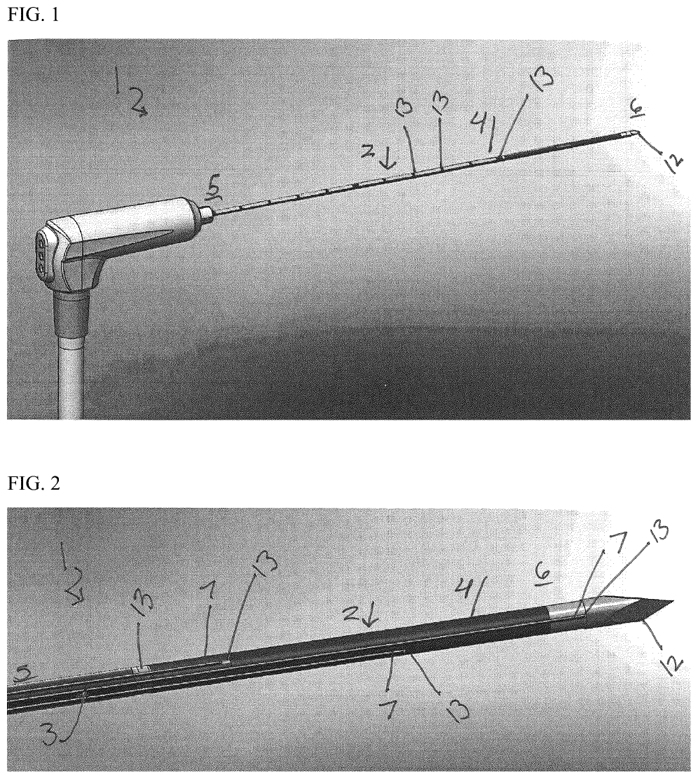

FIGS. 1 and 2 show a temperature regulating device 1 embodiment of the present invention. The temperature regulating device 1 is not limited to a particular design or configuration. In some embodiments, the design or configuration of the temperature regulating device 1 is such that it is able to be positioned at a desired tissue region during medical procedures and measure one or more temperatures. In some embodiments, the temperature regulating device 1 has sufficient flexibility to access a circuitous route through a subject (e.g., through a branched structure, through a bronchial tree, through any region of the body to reach a desired location).

In certain embodiments, as shown in FIGS. 1 and 2, the temperature regulating device 1 has elongate tubular body 2 comprising an elongate tubular body interior region 3 (not shown in FIG. 1), an exterior main body region 4, an elongate tubular body proximal end 5, and an elongate tubular body distal end 6. In some embodiments, the arrangement and positioning of the elongate tubular body interior region 3, the elongate tubular body exterior region 4, the elongate tubular body proximal end 5, and the elongate tubular body distal end 6 within the elongate tubular body 2 is not limited. In some embodiments, the arrangement and positioning of the elongate tubular body interior region 3, the elongate tubular body exterior region 4, the elongate tubular body proximal end 5, and the elongate tubular body distal end 6 within the elongate tubular body 2 is such that it renders the temperature regulating device 1 capable of being positioned at a desired tissue region during medical procedures, measuring temperatures of tissue regions, and reducing the temperature of tissue regions.

Still referring to FIGS. 1 and 2, the elongate tubular body 2 is not limited to a particular composition. In some embodiments, the composition of the elongate tubular body 2 is any composition that renders the temperature regulating device 1 capable of being positioned at a desired tissue region during medical procedures, measuring temperatures of tissue regions, and reducing the temperature of tissue regions. In some embodiments, the composition of the elongate tubular body 2 is a polymer material. In some embodiments, the composition of the elongate tubular body 2 is a higher temperature rated polymer material. Such embodiments are not limited to a particular higher temperature rated polymer material. In some embodiments, the higher temperature rated polymer material is fluorinated ethylene propylene (FEP). In some embodiments, the higher temperature rated polymer material is a thermoplastic copolyester. In some embodiments, the thermoplastic copolyester is Arnitel. In some embodiments, the higher temperature rated polymer material is a fluoropolymer. Such embodiments are not limited to a particular fluoropolymer. In some embodiments, the fluoropolymer is perfluoromethylalkoxy alkane (MFA). In some embodiments, the fluoropolymer is perfluoroalkoxy alkane (PFA). In some embodiments, only a portion (5%, 10%, 25%, 50%, 75%, 77%, 79%, 85%, 88%, 90%, 94%, 98%, 99%, 99.999%) of the elongate tubular body 2 has a composition of a higher temperature rated polymer material. In some embodiments, only a portion (5%, 10%, 25%, 50%, 75%, 77%, 79%, 85%, 88%, 90%, 94%, 98%, 99%, 99.999%) starting from the elongate tubular body distal end 6 has a composition of a higher temperature rated polymer material. In some embodiments, the entire elongate tubular body 2 has a composition of a higher temperature rated polymer material. In some embodiments, the elongate tubular body comprises plastic (e.g., PEEK). In some embodiments, the elongate tubular body comprises thermoplastic polymers that have an appropriate glass-transition temperature of approximately 15-25 degrees Celsius.

Still referring to FIGS. 1 and 2, the elongate tubular body interior region 3 is configured to circulate a coolant for purposes of regulating the temperature (e.g., increasing, maintaining, or reducing) at or near the elongate tubular body exterior region 4. The elongate tubular body interior region 3 is not limited to a particular manner of circulating a coolant for purposes regulating the temperature (e.g., increasing, maintaining, or reducing) at or near the elongate tubular body exterior region 4. In some embodiments, as shown in FIG. 2, the elongate tubular body interior region 3 is able to circulate a coolant through use of one or more coolant channels 7 (FIG. 2 shows three coolant channels 7).

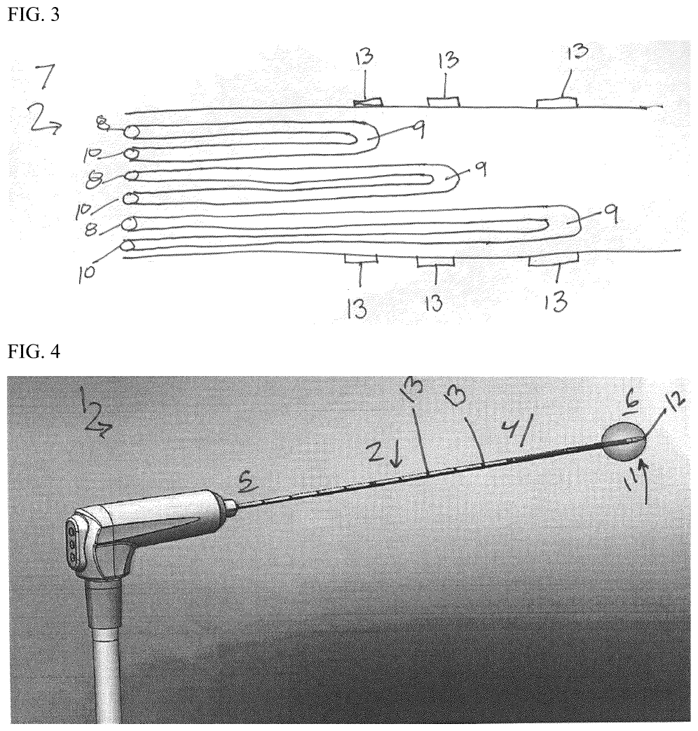

FIG. 3 shows a coolant channel 7 embodiment. As shown, in some embodiments, the elongate tubular body interior region 3 is configured to a) receive coolant into the elongate tubular body proximal end 5 via a coolant intake channel 8, b) circulate the received coolant through the coolant intake channel 8 to the coolant channel contained region 9, and c) circulate the coolant from the coolant channel contained region 9 through the coolant outtake channel 10 and out of the elongate tubular body proximal end 5.

Still referring to FIG. 3, the coolant intake channel 8 and coolant outtake channel 10 are not limited to particular sizes. In some embodiments, the diameter of the coolant outtake channel 10 is larger than the diameter of the coolant intake channel 8. In some embodiments, the diameter of the coolant outtake channel 10 is smaller than the diameter of the coolant intake channel 8. In some embodiments, the diameter of the coolant outtake channel 10 and the diameter of the coolant intake channel 8 are identical.

Still referring to FIG. 3, the coolant channel contained region 9 is not limited to a particular size. In some embodiments, the size of the coolant channel contained region 9 is such that it is able to accommodate coolant circulated through the coolant intake channel 8 for purposes of regulating the temperature (e.g., increasing, maintaining, or reducing) at or near the entire elongate tubular body exterior region 4 or a specific location on the elongate tubular body exterior region 4.

Referring to FIGS. 2 and 3, the coolant channels 7 are not limited to a particular composition. In some embodiments, the coolant channels 7 are plastic (e.g., PEEK). In some embodiments, the coolant channels 7 are metal. In some embodiments, the coolant channels 7 are flexible. In some embodiments, the coolant channels 7 are rigid. In some embodiments, the coolant channels 7 are a mixture of metal and plastic.

Such embodiments are not limited to use of a specific type or kind of coolant. In some embodiments, the coolant is selected from water, glycol, air, inert gasses, carbon dioxide (CO₂), nitrogen, helium, sulfur hexafluoride, ionic solutions (e.g., sodium chloride with or without potassium and other ions), dextrose in water, Ringer's lactate, organic chemical solutions (e.g., ethylene glycol, diethylene glycol, or propylene glycol), oils (e.g., mineral oils, silicone oils, fluorocarbon oils), liquid metals, freons, halomethanes, liquified propane, other haloalkanes, anhydrous ammonia, sulfur dioxide, and a coolant gas compressed at or near its critical point. In some embodiments, the coolant channels are configured to circulate a pressurized gas at zero to 1000 psi.

Referring to FIGS. 2 and 3, the coolant channels 7 are not limited to a particular positioning within the elongate tubular body interior region 3. In some embodiments, the coolant channels 7 are positioned to regulate the temperature (e.g., increasing, maintaining, or reducing) at or near the entire elongate tubular body exterior region 4. In some embodiments, the coolant channels 7 are positioned to regulate the temperature (e.g., increasing, maintaining, or reducing) at or near a specific location of the elongate tubular body exterior region 4. In some embodiments, the coolant channels 7 are positioned to decrease the temperature at or near a location of elongate tubular body exterior region 4 determined to have a temperature above a desired temperature. In some embodiments, each separate coolant channel 7 is positioned to decrease the temperature at or near a specific location of elongate tubular body exterior region 4 determined to have a temperature above a desired temperature.

In some embodiments, each separate coolant channel 7 is positioned to decrease the temperature at or near a specific location of elongate tubular body exterior region 4 determined to have a temperature above a desired temperature (e.g., at or near a temperature sensor). In some embodiments, each separate coolant channel 7 is paired with a temperature sensor such that the coolant channel is able to decrease the temperature at or near a specific location of elongate tubular body exterior region 4 having a temperature sensor.

FIG. 4 shows a temperature regulating device having a cooling zone 11 generated by a coolant channel 7 (not shown) configured to decrease the temperature at or near a specific location of elongate tubular body exterior region 4.

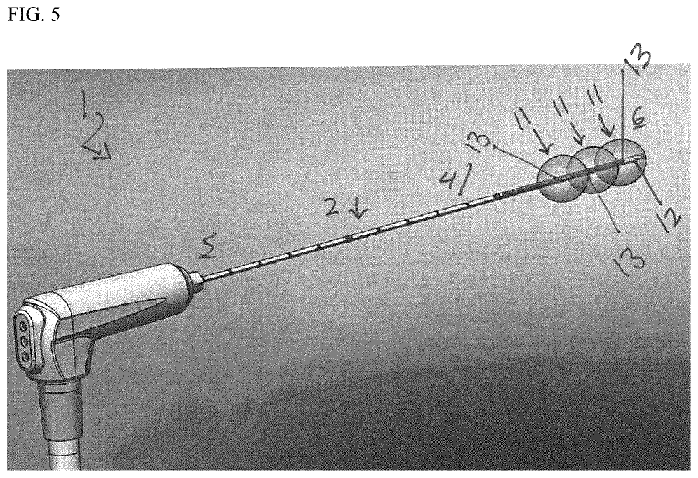

FIG. 5 shows a temperature regulating device having three overlapping cooling zones 11 generated by three separate coolant channels 7 (not shown) configured to decrease the temperature at or near a specific location of elongate tubular body exterior region 4.

Referring to FIGS. 1, 2, 4, and 5, in some embodiments, the temperature regulating device 1 has thereon a stylet tip 12. In some embodiments, the stylet tip 12 is positioned (secured, attached) at the elongate tubular body distal end 6. In some embodiments, the stylet tip 12 is sharp. In some embodiments, the stylet tip 12 is sharp such that capable of insertion through a tissue region (e.g., through skin, through an internal body region (e.g., through an organ, bone, etc.). In some embodiments, the stylet tip 12 is sharp such that capable of percutaneous insertion. The stylet tip 12 may be made of any material. In some embodiments, the stylet tip 12 is made from hardened resin. In some embodiments, the stylet tip 12 is metal.

Referring to FIGS. 1-5, the exterior main body region 4 has thereon one or more (e.g., 1, 2, 3, 4, 5, 6, 7, 8, 9, 10, 15, 20, 25, 50, etc.) temperature sensors 13. In some embodiments, the temperature sensors 13 are designed to measure the temperature of, for example, the exterior main body region 4 and/or a tissue region contacting the energy delivery device in the vicinity of the temperature sensor 13. Such embodiments are not limited to a particular type or kind of temperature sensor 13. In some embodiments, the temperature sensor 13 is a thermocouple. In some embodiments, the one or more temperature sensors comprise a temperature sensor array (e.g., Fiber Bragg Grating optical fiber sensor).

Such embodiments are not limited to a particular positioning of the temperature sensor 13 along the exterior main body region 4. In some embodiments, each temperature sensor 13 is linearly positioned along the length of the exterior main body region 4. In some embodiments, each temperature sensor 13 is wrapped around the entirety of the exterior main body region 4. In some embodiments having two or more temperature sensors 13, each temperature sensor 13 is separated along the length of exterior main body region 4 by at least two centimeters.

In some embodiments, the temperature sensors 13 are configured to wirelessly provide information (e.g., measured temperatures) to a processor. In some embodiments, the temperature sensors 13 are configured to wirelessly provide information (e.g., measured temperatures) to a user. In some embodiments, the temperature sensors 13 are configured to measure a temperature upon direction by a user. In some embodiments, the temperature sensors 13 are configured for continuous temperature measurement.

In some embodiments, the temperature regulating devices are operated manually (e.g., by a user).

In some embodiments, the temperature regulating devices are operated by a processor. The present invention is not limited to a particular type of processor. Such embodiments are not limited to a particular type of processor. In some embodiments, the processor is designed to, for example, receive information from the temperature regulating devices (e.g., the temperature sensors), display such information to a user, and manipulate (e.g., control) the temperature regulating devices.

In some embodiments, the processor comprises software that, when executed, causes the processor to manually or automatically:
process temperature information received from the one or more temperature sensors,
identify temperature sensors measuring a temperature of the exterior elongate tubular body region in the vicinity of the specific temperature sensor to be above a pre-determined level,
identify temperature sensors measuring a temperature of the exterior elongate tubular body region in the vicinity of the specific temperature sensor to be at or below a pre-determined level, and
direct the device to reduce the temperature of the exterior elongate tubular body region in the vicinity of a specific temperature sensor identified as measuring a temperature of the exterior elongate tubular body region in the vicinity of the specific temperature sensor to be above a pre-determined level.

In some embodiments, the processor comprises software that, when executed, causes the processor to manually or automatically:
process temperature information received from the one or more temperature sensors,
identify temperature sensors measuring a temperature of the exterior elongate tubular body region in the vicinity of the specific temperature sensor to be above a pre-determined level,
identify temperature sensors measuring a temperature of the exterior elongate tubular body region in the vicinity of the specific temperature sensor to be at or below a pre-determined level,
direct the device to reduce the temperature of the exterior elongate tubular body region.

In some embodiments, the processor comprises software that, when executed, causes the processor to manually or automatically direct the device to reduce the temperature of the exterior elongate tubular body region.

In some embodiments, the temperature regulating devices further contain a steerable pull ring. Such embodiments are not limited to a particular configuration for the steerable pull ring. In some embodiments, the steerable pull ring has any configuration that permits a user to manually steer the temperature regulating devices via manipulation of the steerable pull ring (e.g., manipulation of one or both of the wires results in a curving or steering of the temperature regulating devices).

In some embodiments, the steerable pull ring permits the temperature regulating devices to be steered in any desired manner or direction. For example, in some embodiments, the steerable pull ring permits the temperature regulating devices to be steered at any desired curve angle (e.g., from 1 to 180 degrees). In some embodiments, the steerable pull ring permits the temperature regulating devices to be steered at any desired bend angle (e.g., from 1 to 360 degrees). In some embodiments, the steerable pull ring permits the temperature regulating devices to be steered at any desired bend radius (e.g., from 1 to 360 degrees). In some embodiments, the steerable pull ring permits the temperature regulating devices to be steered at any desired curve diameter. In some embodiments, the steerable pull ring permits the temperature regulating devices to be steered at any desired reach (e.g., from 0.1 to 100 mm). In some embodiments, the steerable pull ring permits the temperature regulating devices to be steered at any desired curl. In some embodiments, the steerable pull ring permits the flexible sheath to be steered at any desired sweep. In some embodiments, the steerable pull ring permits the temperature regulating devices to be steered at any desired curve (e.g., symmetrical or asymmetrical) (e.g., multi-curve or compound curve). In some embodiments, the steerable pull ring permits the temperature regulating devices to be steered at any desired loop. In some embodiments, the steerable pull ring permits the temperature regulating devices to be steered at any desired deflection (e.g., on-plane deflection, off plane deflection).

In some embodiments, the present invention provides systems for medical procedures providing the temperature regulating devices described herein, and one or more of a processor as described herein, a coolant supply in communication with the temperature regulating devices, a power supply, and a medical procedure device (e.g., a microwave ablation device) (e.g., any type or kind of ablation device) (e.g., any type of kind of medical procedure device that has the potential when operated to unintentionally burn tissue regions).

In some embodiments, the present invention provides systems for medical procedures providing the temperature regulating devices described herein, and one or more of a processor as described herein, a coolant supply in communication with the temperature regulating devices, a power supply, and an energy delivery device. Such embodiments are not limited to a particular type or kind of energy delivery device (e.g., ablation device, surgical device, etc.) (see, e.g., U.S. Pat. Nos. 7,101,369, 7,033,352, 6,893,436, 6,878,147, 6,823,218, 6,817,999, 6,635,055, 6,471,696, 6,383,182, 6,312,427, 6,287,302, 6,277,113, 6,251,128, 6,245,062, 6,026,331, 6,016,811, 5,810,803, 5,800,494, 5,788,692, 5,405,346, 4,494,539, U.S. patent application Ser. Nos. 11/728,460, 11/728,457, 11/728,428, 11/237,136, 11/236,985, 10/980,699, 10/961,994, 10/961,761, 10/834,802, 10/370,179, 09/847,181; Great Britain Patent Application Nos. 2,406,521, 2,388,039; European Patent No. 1395190; and International Patent Application Nos. WO 06/008481, WO 06/002943, WO 05/034783, WO 04/112628, WO 04/033039, WO 04/026122, WO 03/088858, WO 03/039385 WO 95/04385; each herein incorporated by reference in their entireties). Such energy delivery devices are not limited to emitting a particular kind of energy. In some embodiments, the energy delivery devices are capable of emitting radiofrequency energy. In some embodiments, the energy delivery devices are capable of emitting microwave energy. Such devices include any and all medical, veterinary, and research applications devices configured for energy emission, as well as devices used in agricultural settings, manufacturing settings, mechanical settings, or any other application where energy is to be delivered.

The temperature regulating devices and related systems are not limited to particular uses. Indeed, such devices and systems of the present invention are designed for use in any setting wherein temperature regulation is applicable. For example, the temperature regulating devices and systems find use for open surgery, percutaneous, intravascular, intracardiac, intraluminal, endoscopic, laparoscopic, or surgical delivery of energy. Such uses include any and all medical, veterinary, and research applications. In addition, the systems and devices of the present invention may be used in agricultural settings, manufacturing settings, mechanical settings, or any other application where energy is to be delivered.

The present invention is not limited by the nature of the target tissue or region. Uses include, but are not limited to, treatment of heart arrhythmia, tumor ablation (benign and malignant), control of bleeding during surgery, after trauma, for any other control of bleeding, removal of soft tissue, tissue resection and harvest, treatment of varicose veins, intraluminal tissue ablation (e.g., to treat esophageal pathologies such as Barrett's Esophagus and esophageal adenocarcinoma), treatment of bony tumors, normal bone, and benign bony conditions, intraocular uses, uses in cosmetic surgery, treatment of pathologies of the central nervous system including brain tumors and electrical disturbances, sterilization procedures (e.g., ablation of the fallopian tubes) and cauterization of blood vessels or tissue for any purposes. In some embodiments, the surgical application comprises ablation therapy (e.g., to achieve coagulative necrosis). In some embodiments, the surgical application comprises tumor ablation to target, for example, metastatic tumors. In some embodiments, the systems including the flexible sheath described herein are configured for movement and positioning, with minimal damage to the tissue or organism, at any desired location, including but not limited to, the lungs, brain, neck, chest, abdomen, and pelvis. In some embodiments, the systems are configured for guided delivery, for example, by computerized tomography, ultrasound, magnetic resonance imaging, fluoroscopy, and the like. Indeed, in some embodiments, all inserted components of such a system are configured for movement along a narrow and circuitous path through a subject (e.g. through a branched structure, through the bronchial tree, etc.).

In certain embodiments, the present invention provides methods of treating a tissue region, comprising providing a tissue region and a system described herein (e.g., a temperature regulating device, and an energy delivery device (e.g., a microwave ablation catheter), and at least one of the following components: a processor, a power supply, an imager, a tuning system, a temperature reduction system, and/or a device placement system; positioning a portion of the energy delivery device in the vicinity of the tissue region, positioning a temperature regulating device in the vicinity of the tissue region, delivering an amount of energy with the device to the tissue region, and regulating the temperature of the tissue region in the vicinity of the temperature regulating device as needed. In some embodiments, the tissue region is a tumor. In some embodiments, the delivering of the energy results in, for example, the ablation of the tissue region and/or thrombosis of a blood vessel, and/or electroporation of a tissue region. In some embodiments, the tissue region comprises one or more of the lung, heart, liver, genitalia, stomach, lung, large intestine, small intestine, brain, neck, bone, kidney, muscle, tendon, blood vessel, prostate, bladder, and spinal cord.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. A device comprising
an elongate tubular body having an elongate tubular body interior region, elongate tubular body exterior region, an elongate tubular body proximal end, and elongate tubular body distal end;
wherein the elongate tubular body defines an elongate tubular body linear axis of the device extending from the elongate tubular body proximal end to the elongate tubular body distal end;
wherein the elongate tubular body exterior region has thereon one or more temperature sensors, wherein each of the one or more temperature sensors is configured to measure a temperature of the elongate tubular body exterior region in the vicinity of the specific temperature sensor;
wherein the elongate tubular body interior region has therein one or more coolant channels for circulating and recirculating coolant.

2. The device of claim 1, further comprising a stylet tip attached at the elongate tubular body distal end.

3. The device of claim 1, wherein the elongate tubular body is flexible.

4. The device of claim 1, wherein the elongate tubular body comprises plastic (e.g., PEEK).

5-6. (canceled)

7. The device of claim 1,
wherein the one or more coolant channels are configured to reduce the temperature of specific regions of the elongate tubular body exterior region or the entire elongate tubular body exterior region,
wherein each of the one or more coolant channels is separately paired with a separate temperature sensor such that circulation of coolant through the specific coolant channel results in a reduction of temperature of the elongate tubular body exterior region at and/or in the vicinity of the temperature sensor for which it is paired.

8. (canceled)

9. The device of claim 1,
wherein the one or more temperature sensors comprises a first temperature sensor;
wherein the one or more coolant channels comprises a first coolant channel;
wherein the first coolant channel is positioned such that circulation of coolant through the first coolant channel results in a reduction of temperature of the elongate tubular body exterior region at and/or in the vicinity of the first temperature sensor;
or
wherein the one or more temperature sensors comprises a first temperature sensor and a second temperature sensor;
wherein the one or more coolant channels comprises a first coolant channel and a second coolant channel;
wherein the first coolant channel is positioned such that circulation of coolant through the first coolant channel results in a reduction of temperature of the elongate tubular body exterior region at and/or in the vicinity of the first temperature sensor;
wherein the second coolant channel is positioned such that circulation of coolant through the second coolant channel results in a reduction of temperature of the elongate tubular body exterior region at and/or in the vicinity of the second temperature sensor; or
wherein the one or more temperature sensors comprises a first temperature sensor, a second temperature sensor, and a third temperature sensor;
wherein the one or more coolant channels comprises a first coolant channel, a second coolant channel, and a third coolant channel;
wherein the first coolant channel is positioned such that circulation of coolant through the first coolant channel results in a reduction of temperature of the elongate tubular body exterior region at and/or in the vicinity of the first temperature sensor;
wherein the second coolant channel is positioned such that circulation of coolant through the second coolant channel results in a reduction of temperature of the elongate tubular body exterior region at and/or in the vicinity of the second temperature sensor;
wherein the third coolant channel is positioned such that circulation of coolant through the third coolant channel results in a reduction of temperature of the elongate tubular body exterior region at and/or in the vicinity of the third temperature sensor;
or
wherein the one or more temperature sensors comprises a first temperature sensor, a second temperature sensor, a third temperature sensor, and a fourth temperature sensor;
wherein the one or more coolant channels comprises a first coolant channel, a second coolant channel, a third coolant channel, and a fourth coolant channel;
wherein the first coolant channel is positioned such that circulation of coolant through the first coolant channel results in a reduction of temperature of the elongate tubular body exterior region at and/or in the vicinity of the first temperature sensor;
wherein the second coolant channel is positioned such that circulation of coolant through the second coolant channel results in a reduction of temperature of the elongate tubular body exterior region at and/or in the vicinity of the second temperature sensor;
wherein the third coolant channel is positioned such that circulation of coolant through the third coolant channel results in a reduction of temperature of the elongate tubular body exterior region at and/or in the vicinity of the third temperature sensor;
wherein the fourth coolant channel is positioned such that circulation of coolant through the fourth coolant channel results in a reduction of temperature of the elongate tubular body exterior region at and/or in the vicinity of the fourth temperature sensor.

10-12. (canceled)

13. The device of claim 1, wherein the one or more coolant channels is one coolant channel configured to reduce the temperature of the elongate tubular body exterior at one or more locations.

14. The device of claim 1, wherein the one or more coolant channels are configured to circulate a pressurized gas, wherein the pressurized gas is CO₂.

15. (canceled)

16. The device of claim 1, wherein the one or more coolant channels are configured to circulate a pressurized gas at zero to 1000 psi.

17. The device of claim 1, wherein the one or more coolant channels are configured to circulate a liquid, wherein the liquid is water.

18-21. (canceled)

22. The device of claim 1, wherein the one or more temperature sensors are independently selected from a thermocouple and a temperature sensor array.

23. The device of claim 1, wherein the diameter of the device is approximately 3 mm or less.

24-25. (canceled)

26. The device of claim 1, the size of the device is 15 gauge or smaller.

27. The device of claim 1, wherein the one or more temperature sensors are configured to send measured temperature information to a processor.

28. The device of claim 27, wherein the one or more temperature sensors are configured to wirelessly send measured temperature information to a processor.

29. The device of claim 27, wherein the processor comprises software that, when executed, causes the processor to manually or automatically:
process temperature information received from the one or more temperature sensors,
identify temperature sensors measuring a temperature of the elongate tubular body exterior region in the vicinity of the specific temperature sensor to be above a pre-determined level,
identify temperature sensors measuring a temperature of the elongate tubular body exterior region in the vicinity of the specific temperature sensor to be at or below a pre-determined level,
direct the device to reduce the temperature of the elongate tubular body exterior region in the vicinity of a specific temperature sensor identified as measuring a temperature of the elongate tubular body exterior region in the vicinity of the specific temperature sensor to be above a pre-determined level.

30. The device of claim 27, wherein the processor comprises software that, when executed, causes the processor to manually or automatically:
process temperature information received from the one or more temperature sensors,
identify temperature sensors measuring a temperature of the elongate tubular body exterior region in the vicinity of the specific temperature sensor to be above a pre-determined level,
identify temperature sensors measuring a temperature of the elongate tubular body exterior region in the vicinity of the specific temperature sensor to be at or below a pre-determined level,
direct the device to reduce the temperature of the elongate tubular body exterior region.

31. The device of claim 27, wherein the processor comprises software that, when executed, causes the processor to manually or automatically:
direct the device to reduce the temperature of the elongate tubular body exterior region.

32. A system comprising a device of claim 1 and one or more of:
a) a processor comprising software that, when executed, causes the processor to manually or automatically:
process temperature information received from the one or more temperature sensors,
identify temperature sensors measuring a temperature of the elongate tubular body exterior region in the vicinity of the specific temperature sensor to be above a pre-determined level,
identify temperature sensors measuring a temperature of the elongate tubular body exterior region in the vicinity of the specific temperature sensor to be at or below a pre-determined level,
direct the device to reduce the temperature of the elongate tubular body exterior region in the vicinity of a specific temperature sensor identified as measuring a temperature of the elongate tubular body exterior region in the vicinity of the specific temperature sensor to be above a pre-determined level;
b) a processor comprising software that, when executed, causes the processor to manually or automatically:
direct the device to reduce the temperature of the elongate tubular body exterior region;
c) a processor comprising software that, when executed, causes the processor to manually or automatically:
process temperature information received from the one or more temperature sensors,
identify temperature sensors measuring a temperature of the elongate tubular body exterior region in the vicinity of the specific temperature sensor to be above a pre-determined level,
identify temperature sensors measuring a temperature of the elongate tubular body exterior region in the vicinity of the specific temperature sensor to be at or below a pre-determined level,
direct the device to reduce the temperature of the elongate tubular body exterior region;
d) a coolant supply in communication with said device; and
e) a power supply electrically connected to the device.

33. A method, comprising:
providing a system of claim 1;
positioning the device in a tissue region such that the one or more temperature sensors are positioned to measure the temperature of the elongate tubular body exterior region in the vicinity of each specific temperature sensor;
measuring the temperature of the tissue region with the positioned device;
identifying temperature sensors measuring a temperature of the elongate tubular body exterior region in the vicinity of the specific temperature sensor to be above a pre-determined level,
directing the device to reduce the temperature of the elongate tubular body exterior region.

34-37. (canceled)
